(19) United States
(12) Patent Application Publication
(10) Pub. No.: US 2010/0174562 A1
(43) Pub. Date: Jul. 8, 2010

(54) WARRANTY METHOD AND SYSTEM

(76) Inventor: Steven B. Siepser, Paoli, PA (US)

Correspondence Address: WOODCOCK WASHBURN LLP, CIRA CENTRE, 12TH FLOOR, 2929 ARCH STREET, PHILADELPHIA, PA, 19104-2891, US

(21) Appl. No.: 12/724,939

(22) Filed: Mar. 16, 2010

Related U.S. Application Data
(63) Continuation of application No. 12/234,272, filed on Sep. 19, 2008, now abandoned.
Application No. 12/234,272 is a continuation of application No. 10/247,945, filed on Sep. 20, 2002, now abandoned.
(60) Provisional application No. 60/323,561, filed on Sep. 20, 2001.

(51) Int. Cl. G06Q 10/00; G06Q 50/00; G06Q 90/00; A61F 9/008

(52) U.S. Cl. 705/3; 705/302; 705/500; 606/5

(57) ABSTRACT

The present invention compensates loss resulting from unexpected outcome of service or unanticipated product performance. A consumer is provided an option to purchase a warranty prior to or during product purchase or service engagement, the warranty offering pre-determined levels of award calculated from a probabilistic, multi-variable process. Each level of award is associated with a warranty purchase price and a degree of variation of actual outcome from expected outcome. For medical services, variables could include age and gender of the patient, skill level and history of the doctor, quality of the hospital, difficulty of the procedure, type of medical equipment used, prior condition of the patient, geographic location and patient profile. The warranty could require the physician to provide additional medical or surgical care, without charge, to optimize actual outcome prior to granting an award.

### CROSS REFERENCE TO RELATED APPLICATIONS

This application claims priority to and is a continuation of U.S. patent application Ser. No. 12/234,272 filed Sep. 19, 2008, which claims priority to and is a continuation of U.S. patent application Ser. No. 10/247,945 filed Sep. 20, 2002, which claims the benefit of U.S. Provisional Application No. 60/323,561, filed on Sep. 20, 2001, the contents of all of which are hereby incorporated herein in their entirety.

### FIELD OF THE INVENTION

The present invention relates generally to warranty programs, and more particularly to a weighted, multi-variable warranty program compensating loss resulting from unexpected outcome of service or unanticipated product performance.

### BACKGROUND

Tort law governs compensation for injury and loss due to adverse occurrences. In medical care, patients seeking compensation or retribution for poor or unexpected medical results must file a malpractice lawsuit. This method of recovery is costly and time consuming for patients, physicians and the legal community. Furthermore, despite numerous awards resulting from malpractice suits, patients with unexpected medical outcomes often do not receive compensation consistent with their degree of incapacity (i.e., the degree of variation of their actual outcome from the expected outcome). A disproportionately large percentage of typical malpractice awards are absorbed by the legal system, while too small a percentage actually compensates the injured for their loss. Moreover, the number of medical malpractice occurrences exceeds the number of malpractice cases filed as risk management programs dissuade patients from seeking redress. On the other hand, many well versed in the legal system receive awards far exceeding, and bearing no relationship to, their degree of incapacity. Accordingly, the present system is cumbersome and labor intensive, and has resulted in little justice with awards having little correlation with the true disability of the injured party.

One underlying problem facing the tort recovery system generally, and medical malpractice particularly, is the degree to which a physician must insure against possible untoward results due to unpredictable awards resulting therefrom. Consumers incurring loss and pursuing potential recovery windfalls are not motivated or committed to cooperative participation in the recovery process. A compensation system where the consumer selects the value of loss before product or service engagement (i.e., before injury occurrence) is an improvement over the present system and a significant step towards tort reform. Furthermore, a compensation system where the consumer selects from various, pre-determined compensation values in scaled relationship to degree of incapacity, or degree of variation of actual outcome from expected outcome (i.e., greater incremental incapacity, greater incremental award), places the patient in control, efficiently fixes compensation to amounts satisfactory to the patient and places manageable controls on the overall system.

Another problem facing the present tort recovery system is the mindset indoctrinated in consumers and patients. No matter how sophisticated and well informed the consumer or patient, the mindset has adverse results ultimately being caused by and the responsibility of someone else. The present system precludes the consumer or patient from fully accepting the fact that untoward results are possible, that final outcome might be less than ideal, and that negligence of another might not be the reason for the untoward result.

At present, there is little option to the present tort recovery system. Manufacturers and service providers are motivated to find an alternative. Consumers would benefit from a system having speedy and just awards, where compensation is pre-operatively selected, with value determined by the consumer. Economically, the present system is punitively onerous, especially for service providers practicing in high-risk specialties. The inefficiencies of the present recovery system could eventually limit consumer access to specialty services and products, as providers move away from areas having high insurance costs.

For the foregoing reasons, there exists an urgent need for a recovery system (warranty product) that assigns compensation in a pre-prescribed manner, with funds efficiently directed toward compensating loss, not absorbed for punitive purpose or by administrative/legal fees. There is a need for a system that does not warranty the product or service itself, but allows subscribers (i.e., patients, consumers) to pre-purchase levels of warranty protection based on their judgment of need, where award values are predetermined and based on a degree of variation of actual outcome from expected outcome.

### SUMMARY OF THE INVENTION

The present invention is a warranty of recovery method and system providing awards for unexpected outcomes. The warranty system assigns compensation in a pre-prescribed manner, allowing consumers to pre-purchase levels of protection based on their judgment of need, having award values based on a degree of variation of actual outcome from expected outcome. The warranty program of the present invention provides consumers of products and services with an alternative to tort remedy, an alternative that first works to correct the injury or loss and, if necessary, to provide the consumer with compensation commensurate with the gravity of unexpected outcome or injury—all through a quick, efficient, and economical program.

In one aspect of the present invention, a consumer is offered the option of pre-purchasing the warranty, prior to or during product purchase or service engagement, the warranty compensating loss resulting from the respective service provided or product purchased. The consumer selects from various levels of award, each derived from one or more factors. The warranty provides measurable outcome expectations and a corresponding award if the expected outcome is not achieved.

In another aspect of the invention, the measurable outcome expectations are incrementally scaled based on a degree of variation of actual outcome from expected outcome.

In another aspect of the invention, an independent advisory board determines the level of incapacity and subsequent warranty award.

In another aspect of the invention, the consumer has the option of voiding the warranty, and corresponding payment, and seek legal action.

In another aspect of the invention, the warranty involves a medical procedure or surgical event.

In another aspect of the invention, award levels are the result of a probabilistic multi-variable process, where variables are selected from the group consisting of: patient age; patient gender; skill level and history of the doctor; type of medical equipment used; quality of the hospital; difficulty of the procedure; prior condition of the patient; patient profile; geographic location; and warranty cost.

In another aspect of the invention, the patient agrees to receive corrective treatment, including additional surgery if needed, to achieve a maximum actual result before an award is determined.

In another aspect of the invention, the surgical event is lasik eye surgery.

In another aspect of the invention, certain restrictions govern physician participation in the warranty program. For instance, a physician must sign a participation agreement, agreeing to be bound by provisions of the warranty program. Provisions governing inclusion in the program could include achieving certain credentials, satisfying certain eligibility requirements and establishing certain quality criteria. Participation in the warranty program could also require a specific level of demonstrated competence and quality control achievements. Accordingly, participation in the warranty program would inure marketing benefits to the physician, establishing and indicating a level of quality and capability in the respective field.

It is an object of the present invention to address the inequities of the present tort recovery system with an effective and just program administered at a far lower cost to satisfy a public having little tolerance for less than ideal outcomes.

Another object of the present invention is to create a new paradigm in tort recovery, one having a better economic foundation to meet the needs of the parties in redress of medical shortcomings.

Another object of the present invention is to provide consumers of products and services with security, the consumer assured in knowing that a predetermined level of compensation awaits injury or loss due to adverse product performance or unexpected outcome of service without enduring the adversarial and unpredictable tort litigation process.

Another object of the present invention is to create a program where large sums of money can be redistributed to those deserving of award through a system designed to award money based solely on the gravity of the injury. The removal of the legal system from this exchange leaves excess profits for a return on investment.

Another object of the present invention is to create a program where injury (damage) type, extent of loss and cause of injury is defined and codified for use in establishing multiple levels of award. Each outcome level has an associated code for ease in the administration and management of the program. The codified system (defining the objective, measurable event outcomes and resulting level of award) is used as a standard for administrative management of not only the warranty program, but of the entire respective trade or business, whether or not the warranty event involves outcome of product performance or professional service.

### BRIEF DESCRIPTION OF THE DRAWINGS

For the purpose of illustrating the invention, certain embodiments are shown in the drawings; it being understood, however, that this invention is not limited to the precise arrangements and instrumentalities shown.

FIG. 1 illustrates by flow diagram process steps for a warranty method, in accordance with the present invention; and

FIG. 2 illustrates by flow diagram process steps for a warranty method directed to lasik eye surgery, in accordance with the present invention.

### DETAILED DESCRIPTION OF ILLUSTRATIVE EMBODIMENTS

The present invention is an outcome based warranty method and system comprising a weighted, multi-variable process. The warranty program compensates loss (unexpected outcome) by providing awards to injured individuals or to those having difficulty resulting from product failure.

In one embodiment of the invention, the program is a doctor administered warranty system. A prospective patient is given the option of purchasing a warranty directed to the outcome of a particular treatment or surgery. The cost (purchase price) of the warranty could be included in the price of the medical treatment or an additional charge paid by the patient. The resulting contractual relationship is guided by a system of registration and possible awards based on a defined codified award system based on results.

In another embodiment of the invention, the program is a product-based warranty, providing award for unexpected outcome resulting from adverse consequences of product use. Injury type, extent of loss and cause of injury would be defined and codified for use in establishing multiple levels of award. In this embodiment, each outcome level receives a code for ease in the administration and management of the warranty program. The codified system (defining the objective, measurable event outcomes and resulting level of award) becomes a standard for administrative management of not only the warranty program, but of the entire respective trade or business, whether or not the warranty event involves outcome of product performance or professional service.

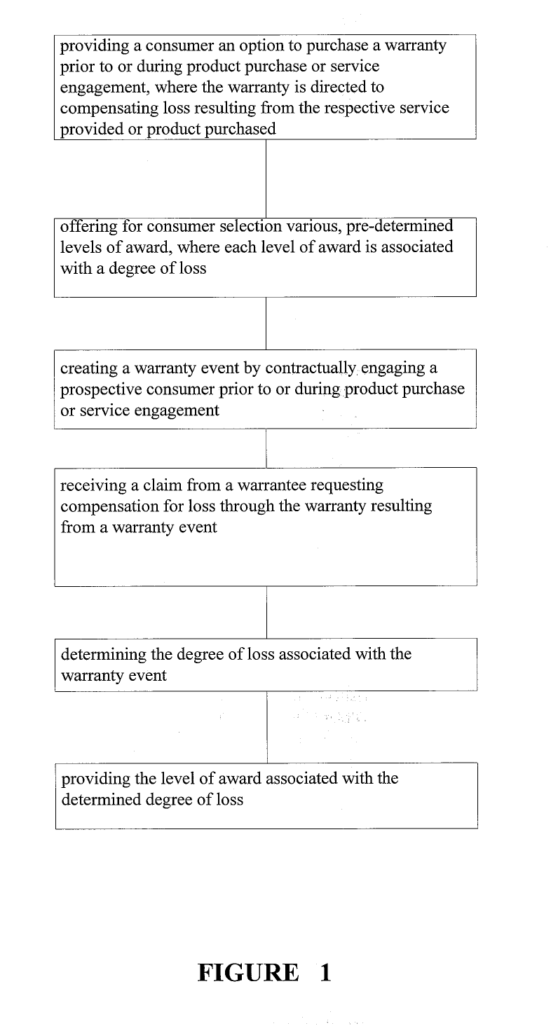

In another embodiment of the invention, the program is a service-based warranty, providing award for unexpected outcome of service falling outside the bounds of a pre-determined range of risk. FIG. 1 illustrates by flow diagram process steps which could be included in almost any embodiment of the invention, whether the warranty is directed to product performance or service outcome.

### The Warranty Process

In one particular embodiment of the invention, the warranty operates as follows. A prospective patient is given the option of participating in the warranty program by purchasing a warranty from a participating physician. The warranty provides measurable outcome expectations including a corresponding award if the expected outcome is not achieved. Under this specific embodiment of the warranty program, the patient must agree to cooperate with physician provided corrective treatment, including additional surgery if necessary, to achieve maximum outcome. Following treatment, an independent advisory board determines the level of disability and subsequent warranty award. In this specific embodiment, a patient cannot abandon a professionally recommended treatment process or refuse professionally recommended additional surgery and still collect an award. In this embodiment of the invention, a patient retains the option of voiding the warranty, and corresponding award, and retains the right to seek legal action.

In another embodiment of the invention, there are two methods for sale of the warranty product. One is a doctor direct sale and another is a third-party sale. The doctor direct warranty is sold to the patient during medical procedure introduction and counseling (prior to service engagement). The doctor direct warranty might require that the doctor qualify for the program.

In still another embodiment of the invention, an important element of the warranty program is the information technology system supporting the sale. The registered physician practice (remote site) must access the active site (warranty program control center) for patient inscription. The patient's propensity to sue and a profile of their behavior is linked with the warranty transaction to assist the physicians' practice and/or the control center in deciding to contractually engage the patient in a warranty.

In yet another embodiment of the invention, a warranty management entity operates the program control center and provides services including: (1) coordinating warranty related services and payments to patients; (2) physician credentialing; (3) warranty program sales, marketing and accounting; (4) physician and program quality assurance; and (5) warranty cost and award payment administration. The management entity administratively pays all awards; physicians have no direct responsibility or personal exposure related to warranty awards made under the program. Further, a database is maintained of physician and patient attributes, along with numerous data elements for each warranty issued (e.g., procedure data, physician data, patient profile, pre-procedure capabilities of patient, post-procedure outcome data).

Prior to awarding warranty compensation, another embodiment of the invention requires patients to timely register their request for loss compensation and decide whether to accept the warranty payment as complete compensation or pursue legal action. An independent advisory board decides the level of award. The patient can access legal representation during this phase of the program. Patients are provided appropriate time to seek redress in the traditional mode if they choose not to accept the warranty award. In this embodiment of the invention, patients are not eligible for the warranty award (i.e., the award is forfeited) if the patient chooses to proceed with standard tort resolution.

### Parameters for Initial and Continued Physician Participation in the Warranty Program

Certain criteria are required of physicians before acceptance into the warranty program and approval as direct sellers of the warranty benefit. Representation as a direct selling physician provider of the warranty program provides numerous benefits for the physician. The physician benefits professionally (by promoting quality) and financially (by enhancing market appeal, adding income and lowering malpractice insurance costs). Management of physician providers, and maintenance of established physician criteria, is handled by control center personnel. Table 1 illustrates examples of possible required criteria for initial participation of physician, physician's office, and patient.

TABLE 1
Personal
Licensed physician
Board certified in specific specialty
Specific number of years in practice
Having completed specific number of procedures
Office location specified and/or limited in number
Have certain insurance coverage
Meets specific medical training criteria (e.g. schooling)
Office
Have specified equipment that has met all quality criteria (e.g. specific
type and age of laser)
Have specific number of office staff
Meet office staff training qualifications
Meet other quality provisions (based on chart reviews of prior cases)
Has specific computer technology to support warranty program
Has specific training to sell and administer warranty program at the
physician level
Only accepts patients meeting certain selective criteria directed to general
medical history and specific, objectively measured conditions
Patient Profile
Type of medical treatment or surgery that qualifies for the warranty
program
Meets specified medical/surgical/psychological criteria
Specified condition objectively measured prior to surgery
Specified medical history (e.g. did not already have unsuccessful treatment
elsewhere)
Specified age (e.g. some medical treatment might impose certain age
restrictions)
Specified sex
Level of compliance for pre-op care
History of malpractice suits registered

Part of the physician and program quality assurance responsibility of the warranty management entity is to maintain the quality and performance of the physicians participating in the program. The database maintained by the management entity includes application logic and statistical analysis programming to process physician, office, patient, procedure and claim attributes regarding each warranty event.

Physician, office and patient attributes could include any or all of the data listed in Table 1. Procedure and claim attributes could include statistical data specific to the procedure, data relevant to surgical equipment used, pre-procedure capabilities of patient, post-procedure outcome of patient, statistical post-procedure vs. pre-procedure data, warranty claims initiated, warranty awards paid, outcome of corrective treatment, advisory panel evaluation data for each claim processed.

Report-generating logic processes reports specific to procedure and physician performance relative to overall procedure statistics. Clinically significant statistical trends are cited for evaluation directed to improvement of procedure treatment methods. Significant variations in warranty claims per procedure are cited for program evaluation. Significant variations in warranty claims per physician are cited for physician evaluation. Physicians with low rates of warranty claims might receive lower warranty rates or program bonuses. Physicians with high rates of warranty claims might receive higher warranty rates, might be required to satisfy further training and/or competency criteria, might face a probationary period, or might be refused further participation in the warranty program.

### Physician Benefits for Program Participation

A physician is motivated to participate in the warranty program because the warranty program allows physician resources to shift from costly malpractice liability avoidance to concentration on quality care through superior outcomes to reduce warranty claims and awards. Successful participation in the warranty program may provide reductions in malpractice insurance costs due to a percentage of the practice shifting to pre-arranged warranty coverage. This shift of practice share to the warranty program results in long-term economic benefit for the physician. As mentioned, supra, quality of outcome is monitored and physicians operating above the norm could receive further economic incentives. Table 2 illustrates other physician benefits, which would not be limited to:

TABLE 2
Access Fee: The warranty program could pay participating physicians
access fees for program participation (i.e., making the warranty
available to their patients).
Quality Incentive: The warranty program may institute a quality-based
incentive program to reward physicians based upon surgical outcomes,
claims experience and similar criteria.
Investment Opportunities: Participating physicians may be given an
opportunity to invest in warranty program, possibly on a preferred
basis based on plan performance or other criteria.
Decreased Malpractice: The warranty program hopes to partner with an
insurance company which may offer participating physicians decreased
medical malpractice premiums or policy credits based upon the physicians'
participation in the warranty program and proven successful claims
experience.
Marketing Edge: Participation in the program will be limited to physicians
who have met strict quality control criteria. Accordingly, physicians can
market their participation in the program, subject to set standards to
ensure confidentiality, legal and ethical standards and propriety, to
differentiate them from competitors.

### Example 1

### Corrective Eye Surgery—The Lasik Model

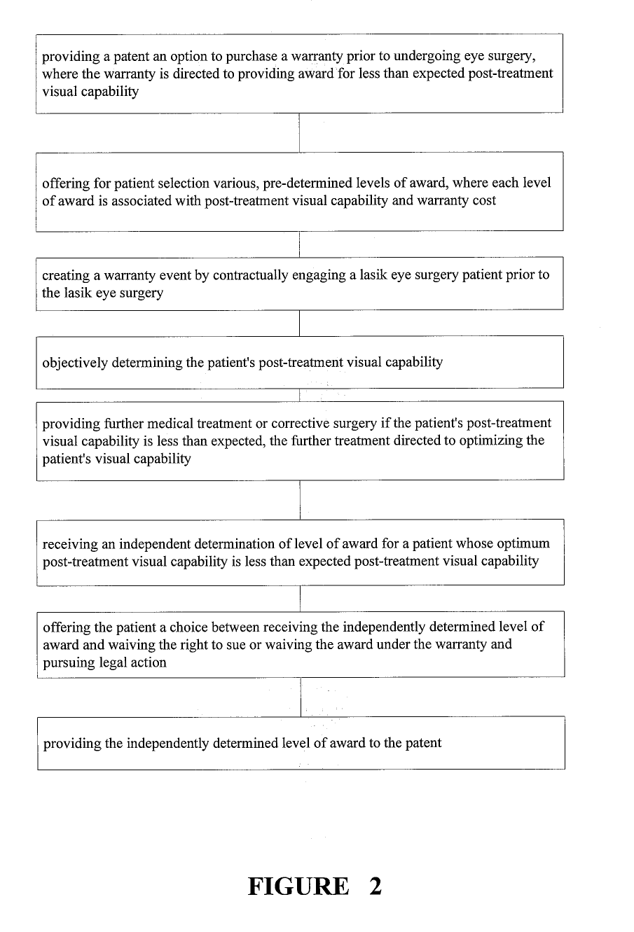

One example of the invention is specifically directed to Lasik eye surgery. FIG. 2 illustrates by flow diagram a summary of process steps included in the Lasik eye surgery embodiment.

Patients electing Lasik procedures presently undergo consultation and counseling sessions related to the surgery. During the initial consultation the suggestion of providing a warranted outcome is mentioned. Once the patient decides to undergo a Lasik procedure, a full disclosure of the warranty program is provided. The counseling sessions related to the eye surgery are secondarily used to screen, inform and contractually engage the patient with the warranty product. In a typical counseling setting, a patient might request a laser evaluation. If the patient meets certain phone criteria (i.e., age, referral status, medical history, phone voice, sincere voice, knowledge base), a counseling session is scheduled. The phone criteria might also act to screen the patient for inclusion in the warranty program.

Before undergoing the laser evaluation, the patient receives an informational counseling session to discuss the procedure and pricing. A full disclosure of the warranty program might also be presented at this counseling session. The patient could receive warranty disclosure from physician and/or staff counseling, videotape viewing or website access.

Subsequent to a decision to undergo Lasik surgery is a time period of at least 24 hours, which allows the patient time to sleep on the decision and consult with legal or other professionals regarding the decision. The warranty also provides ample time to seek legal opinion following execution.

The physician's staff (remote site) visits the web site of a central program controller to enter the patient in the warranty program. Patient data is evaluated, screening the patient for litigation profile and program qualification. Patient profiling could occur at a remote site, or directly from the central controller site.

The program provides a variable warranty based on a table of possible outcomes and a written and oral presentation of data. The patient can review various packages providing for higher awards based on the warranty cost and the severity of the outcome (i.e., degree of variation of actual outcome from expected outcome). The factors used to arrive at a level of award can be divided into areas that consider subjective complaint and objective measurement. The level of award could be a composite resultant of a probabilistic process.

Table 3 illustrates one embodiment of variables used to objectively determine the level of award for lasik eye surgery. In this embodiment, the levels of award are incrementally scaled based on warranty cost and degree of variation of actual outcome (post-surgery eyesight) from expected outcome. Upon electing to enter the warranty program, the patient selects the level of warranty cost.

TABLE 3

| | | |
|---|---|---|
| Bronze | Gold | Platinum |

WARRANTY SELECTION

| | | | |
|---|---|---|---|
| Warranty Cost | $50 | $100 | $150 |

AWARDS

| | | | |
|---|---|---|---|
| BCVA# BCVA best | $10,000/eye | $20,000/eye | $30,000 |
| corrected visual acuity# | | | |
| 20/50 | | | |
| BCVA 20/100 | 20,000 | 40,000 | 60,000 |
| BCVA 20/200 | 50,000/eye | 100,000/eye | 150,000 |
| Legal blindness | | | |
| Irreversible loss of | 100,000/eye | 150/000/eye | 300,000 |
| vision | | | |
| Loss of an eye | 200,000/eye | 300,000 | 400,000 |
| Bilateral legal blindness | 500,000 | 750,000 | 1,000,000 |

Upon completion of the lasik procedure, a post-surgery examination is conducted to obtain objective outcome measurements. If actual outcome is less than expected outcome, the physician determines if further treatment or corrective surgery could place the patient closer to expected outcome. A release time is established within which all corrective treatment must occur (i.e., the physician feels all that can be done has been done).

A patient cannot abandon the treatment process or refuse additional surgery to collect an award. This policy assures that patients achieve their maximum function, or best possible outcome. This policy is consistent with success, and a cooperative patient is better motivated to achieve success.

After exhaustion of corrective treatment, the operating physician certifies that nothing further can improve outcome. A board member, or independent advisory expert, examines the patient and determines whether an award is the best solution, or in the best interest of the patient. The major objective is the assurance of the highest quality of care with awards for untoward outcomes without relying on the present cumbersome and unbalanced system of disproportionate awards. As mentioned, supra, a secondary objective is policing the participating physicians; doctors with poor outcomes can be objectively identified without being stigmatized by the questionable measure of number of lawsuits filed.

Table 4 illustrates another embodiment of the warranty program, including limited and specific variables used to objectively determine the level of award for lasik eye surgery, along with specific restrictions (i.e., requirements) for program qualification, the specific restrictions providing for maximum level of award. It shall be recognized, however, that various levels of award are possible and could be offered for any one or all of the incremental levels of post-surgery eyesight illustrated in Tables 3 and 4, depending upon careful selection and application of one or more of the specific restrictions listed in Table 4, or the general restrictions listed in Table 1. Additionally, further increments of post-surgery eyesight are feasible (i.e., additional table entries of incremental outcomes, each with an associated award amount). Further restrictions for program qualification are also feasible, the restrictions directed to physician, patient, center, the general program, or a combination thereof.

TABLE 4
WARRANTY SELECTION

| | |
|---|---|
| Warranty Cost | $125 per eye |

AWARDS

| | |
|---|---|
| BCVA - Best Corrected Visual Acuity of | $500,000 |
| 20/200 or worse (Legal blindness | |
| in one eye) | |
| BCBV - Best Corrected Binocular Vision of | $250,000 |
| 20/50 or worse | |
| (both eyes combined) | |
| Loss of an eye | $1,000,000 |

Office (Center) Requirements for Program Participation
An approved microkeratome must be used (e.g., BD-3000,
Hansatome, Amadeus, BKM, Nidek)
An approved Laser must be used (will change over time)
VISX 3 Scanning Laser
Autonomous LADAR Vision
Technolas 217, Bausch and Lomb (myopia −1.0-−7.0 D)
An automated pupillometer must be used
No “roll on, roll off” lasers may be used
No mobile lasers may be used
Only fixed lasers may be used
Defensive Risk Management Procedures in place
Patient Psychometric Testing
Occupational Screening
Standardized Counseling
Background Check
General Program Requirements
Award provided after patient has undergone all medically
appropriate treatment, including additional Lasik procedures (i.e.,
enhancements) if the initial procedure does not meet final goals of
patient and physician
Patients reviewed by Program Board to evaluate eligibility
Patient is eligible to collect only one award

Wavefront analysis can be used to objectively measure visual performance. Wavefront analysis will prevent a patient from feigning poor vision in an attempt to fraudulently profit from the warranty program. Wavefront analysis is a computerized method, which uses microchip arrays to determine whether an image projected on the back of the eye is in focus. Wavefront analysis objectively measures resolution of the eye, and can determine whether the image projected on the back of the eye includes optical aberrations such as blur, glare or loss of contrast sensitivity.

The embodiment of the warranty program illustrated in Table 4 includes awards for only three objectively measured variables (i.e., post-surgery eyesight conditions). The Table 4 warranty program embodiment does not include negative outcomes and complications such as reduced night vision, ghosting (a fainter second image of an object is seen), halos (lights appear as glare or surrounded by rings), starbursts (bright lights are seen as spiked circles of light), double vision (two overlapping images are seen at the same time), dry-eye syndrome (the eyes do not produce enough tears to stay moist and comfortable), loss of contrast vision, corneal edema, corneal scarring, persistent epithelial defect, epithelial abrasion, interface epithelium, cap thinning, interface debris, epithelial ingrowth, infectious keratitis, microbial keratitis, pain, diffuse lamellar keratitis, vitreous hemorrhage, retinal detachments, and loss of vision that occurs from other sources including trauma to the eye after the surgery. It shall be recognized, however, that further embodiments of the present invention could include awards for any or all of the negative outcomes and complications listed above, along with other less objective (or even subjective) negative outcomes.

For example, the present invention could include coverage for reduced night vision and complications due to glare. Variables used to determine eligibility for award could include pre-determined results based on one or more of the following tests:
1) Contrast testing performed in the three wavelengths. Patient does not must return to pre-operative reading ability in 1 year, within 2 standard deviations.
2) Loss of ability to perform job requirements
3) Post-operative and Pre-operative comparison of night driving evaluation (IOWA correlation with Glarometer testing).
4) Post-operative and Pre-operative comparison of Glarometer testing, with patient having more than a two line increase in glare measurement of either or both of starburst and halo.
5) Distance evalution descriptions
6) Multiple contrast testing.
7) Diplopia showing presence with loss of all stereo vision
8) Abnormal Worth 4 dot test
9) Frank esotropia or exotropia on cross cover test
   Variables addressing coverage for reduced night vision and complications due to glare could include one or more of the restrictions included in Table 1 and 4 directed to physician, patient, center, the general program, or a combination thereof.

The patient is allowed one month to elect to receive the award under the program. If elected, the patient is then notified of the level of award. The patient receives 5 business days to accept the award and waive the right to sue. Alternatively, the patient could choose not to collect the award, forfeit the warranty fee paid and pursue legal action.

### Example 2

### Investment Services

In the investment services area, an individual seeking assistance with a financial planning service could purchase a warranty with a clarified risk. A warranty program could help stem the present number of lawsuits occurring after a downturn in the stock market or in new investment vehicles. The consumer could draw from a pooled resource if there was an untoward outcome from a particular investment.

### Example 3

### Blood Transfusions

Blood transfusion is a routine medical procedure lending itself to the warranty program because clear award criteria (i.e., objectively measured outcomes) can be established. An individual requiring blood could purchase a warranty assuring that the blood received is correct and untainted. One level of award could exist for receiving the wrong blood. Greater awards could be associated with certain adverse reactions and another award if death occurs.

### Example 4

### Cardiac Surgery

Bypass surgery is a less typical medical procedure, but one that could be protected by the warranty program because objective, before and after performance can be determined. First, pre-operation limitations are measured. Failure to exceed these after surgery would result in a level of award. A positive stress test or less then 125% performance on retesting could result in another award. Further awards could be incrementally scaled based on echocardiograph measurements of blood flow after surgery. Awards could extend to total cardiac disability and death.

### Example 5

### Orthopedic Surgery

Orthopedic surgery (e.g., joint replacement) lends itself to functional testing. Knees seem to be a good demonstration of the comment. Levels of award could incrementally track the degree of improvement, or lack thereof, in flexibility, weight bearing ability, and complete range of motion. MRI of the subject joints could show functionality and structure.

### Example 6

### Airplane Purchase

An injury per seat warranty could accompany the purchase of an airplane. If injury occurs in the airplane, for whatever reason or cause, a level of award is paid. Injury type, extent of loss and cause of injury define the level of award in this embodiment. The warranty is product-based, the individual purchasing the airplane receives a warranty on use of the airplane and is compensated for any injury or untoward outcome occurring from such use. Naturally, this embodiment of the invention could easily be modified to cover cars, motorcycles, boats or other means of conveyance.

### General Discussion

The warranty program is product-based, which is one factor distinguishing the present invention from accidental death, dismemberment or other types of injury insurance. The warranty program of the present invention is also differentiated from standard product warranties, which warrant only the product and not unexpected outcome resulting from use thereof. Currently, recovery for injury or loss resulting from product use requires a lawsuit. The warranty program of the present invention provides a level of award prescribed prior to the occurrence of injury or loss (i.e., at product purchase) and compensates individuals who are injured or have difficulty as a result of a product's failure to perform.

Presently, costs of liability insurance are responsible for a significant percentage of the purchase price of many consumer products, especially airplanes. These significant insurance margins, much of which are absorbed by the administrative, legal and punitive costs of tort litigation, could be reduced or eliminated if a consumer instead purchased a warranty covering product performance and compensating any injury or untoward outcome resulting from product use.

Although punitive aspects of tort litigation can promote design and/or procedural change to improve safety, the present costs of doing so are extreme and, therefore, cumbersome and inefficient. The warranty program of the present invention promotes safety in manufacturing by motivating the manufacturer to strive for lower warranty rates, as warranty rates are determined by tracking awards and the reasons behind award payments. In addition, consumers could be provided statistical information compiled by the warranty program regarding rates of accident, injury or problem related to individual product.

Replacing the inefficiencies (i.e., large percentage of awards going to legal services and serving punitive purposes) of the present system with just and efficient compensation provided through the warranty program of the present invention leaves a large margin (even after the injured party receives a significant award) to serve other purposes. Portions of this margin could be used to lower product or service cost, research improvements to the product or service or serve as profits for the warranty program. Warranty program profits could be directed to lowering warranty costs for lower risk manufacturers and service providers. Risk determinations would be determined by warranty program statistics. A lower risk classification, as determined by the warranty program, would provide manufacturers and service providers with an incentive to improve products/services and assure quality. A lower risk classification would also serve to distinguish quality to potential consumers.

Apparent from the above description of the present invention is that the warranty program is applicable to, and can be developed for, many product purchase or service engagement situations. Any situation where injury could occur or loss could result, from a product's failure to perform as expected, or from less than expected outcome of service, is ripe for application of the warranty program of the present invention. From an administrative standpoint, any situation where objective discriminate variables can be developed, as a measure of outcome or quality for a specific event, lends itself to warranty program application. More particularly, products and services having low risk (i.e., low incidence of poor outcome) and high volume are the best model, as are single procedures and elective self-pay procedures in areas requiring high expertise.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention, including awards for subjectively measurable variable conditions.

What is claimed:

1. A method for compensating loss resulting from unexpected outcome of service or unanticipated product performance, comprising the steps of:
a. providing an option to purchase a warranty prior to or during product purchase or service engagement, the warranty compensating loss resulting from the respective service provided or product purchased;
b. offering various, pre-determined levels of award with the warranty, each level of award associated with a degree of loss, wherein a warranty purchase creates a warranty event;
c. determining the degree of loss associated with the warranty event; and
d. providing the level of award associated with the determined degree of loss.

2. The method of claim 1, wherein the level of award is derived from at least one factor.

3. The method of claim 2, wherein the level of award results from a probabilistic, multi-variable process.

4. The method of claim 1, wherein the degree of loss is based on measurable outcome expectations.

5. The method of claim 4, wherein the measurable outcome expectations are incrementally scaled and the degree of loss is based on a degree of variation of actual outcome from expected outcome.

6. The method of claim 1, wherein an independent advisory board determines the degree of loss.

7. The method of claim 1, wherein a warrantee has an option to void the warranty after the degree of loss is determined, forfeit the award and pursue other methods of recovery.

8. The method of claim 1, wherein a warrantee has an option to void the warranty after the warranty is purchased and pursue other methods of recovery.

9. The method of claim 1, wherein the respective service provided involves a medical procedure or surgical event.

10. The method of claim 9, wherein the level of award results from a probabilistic, multi-variable process.

11. The method of claim 10, wherein the variables are selected from the group consisting of patient age, patient gender, skill level and history of the doctor, quality of the hospital, type of medical equipment used, difficulty of the procedure, prior condition of the patient, patient profile, geographic location and warranty cost.

12. The method of claim 5, wherein the respective service provided involves a medical procedure or surgical event.

13. The method of claim 12, wherein the patient agrees to receive corrective treatment to optimize the actual outcome before the degree of loss is determined.

14. The method of claim 12, wherein physician participation requires compliance with certain restrictions.

15. The method of claim 14, wherein the restrictions are selected from the group consisting of executing a participation agreement binding the physician to certain contractual provisions, achieving certain credentials, satisfying certain eligibility requirements, establishing and maintaining certain quality criteria and achieving a specific level of demonstrated competence.

16. The method of claim 1, wherein the respective service provided is lasik eye surgery.

17. A method providing award for unexpected medical outcomes, comprising the steps of:
a. providing an option to purchase a warranty prior to or during engagement of medical treatment, the warranty providing award for unexpected outcome;
b. offering various, pre-determined levels of award with the warranty, each level of award associated with a degree of loss, the degree of loss based on a degree of variation of actual outcome from expected outcome, wherein a warranty purchase creates a warranty event;
c. determining the degree of loss associated with the warranty event; and
d. providing the level of award associated with the determined degree of loss.

18. The method of claim 17, wherein the level of award results from a probabilistic, multi-variable process.

19. The method of claim 18, wherein the variables are selected from the group consisting of patient age, patient gender, skill level and history of the doctor, quality of the hospital, type of medical equipment used, difficulty of the procedure, prior condition of the patient, patient profile, geographic location and warranty cost.

20. The method of claim 17, wherein the patient agrees to receive corrective treatment to optimize the actual outcome before the degree of loss is determined.

21. A method for implementing a warranty program to compensate patients of lasik eye surgery that attain less than expected post-treatment visual capability, comprising the steps of:
a. providing an option to purchase a warranty prior to undergoing lasik eye surgery, the warranty providing award for less than expected post-treatment visual capability;
b. offering various, pre-determined levels of award with the warranty, each level of award associated with post-treatment visual capability, whereby a warranty purchase initiates warranty coverage and award applicability;
c. determining post-treatment visual capability; and
d. providing the level of award associated with the post-treatment visual capability.

22. The method of claim 21, further comprising the step of patient screening, wherein the patient is interviewed and patient data is evaluated for warranty program qualification.

23. The method of claim 22, wherein the patient data used to evaluate whether a patient qualifies for the warranty program includes negative testing to conditions selected from the group consisting of auto-immune disease, diabetes, unstable refractions, change in cylinder over 1.00 D, Axis 15, previous history of dry eye, irregular astigmatism, keratoconus, pregnancy, herpes, uncontrolled glaucoma and BCVA of 20/30 or worse.

24. The method of claim 21, wherein each level of award is also associated with a pre-determined warranty cost.

25. The method of claim 21, wherein a level of award is associated with post-treatment visual capability of one eye or both eyes selected from the group consisting of irreversible loss of vision, loss of the eye, BCVA of 20/50 or worse, BCVA or 20/100 or worse, BCVA or 20/200 or worse and BCBV of 20/50 or worse.

26. The method of claim 21, wherein post-treatment visual capability is determined by wavefront analysis.

27. The method of claim 21, wherein a level of award is associated with post-treatment visual capability directed to reduced night vision and glare complications.

28. The method of claim 27, wherein post-treatment visual capability is determined using a Glarometer.

29. The method of claim 21, wherein providers of the lasik eye surgery must meet or exceed certain qualifications to participate in the warranty program.

30. The method of claim 29, wherein the certain qualifications are selected from the group consisting of provider execution of a participation agreement binding the provider to certain contractual provisions and technical criteria, achieving certain credentials, satisfying certain eligibility requirements, establishing and maintaining certain quality criteria and achieving a specific level of demonstrated competence.

31. The method of claim 30, wherein satisfying certain eligibility requirements includes performing a minimum of 500 Lasik surgeries per year and having performed a minimum of 1,000 Lasik surgeries to date.

32. The method of claim 30, wherein the technical criteria is selected from the group consisting of use of an approved Microkeratome, use of an approved Laser, use of a VISX 3 Scanning Laser, use of Autonomous LADAR Vision, use of Technolas 217, Bausch and Lomb (myopia −1.0-−7.0 D), use of an automated pupillometer, no use of a “roll on, roll off” Laser, no use of a mobile Laser, requirement that the patient's residual bed is greater than 250 microns, requirement that the patient's pupils are under 7 mm (dim light), no patients with myopes greater than −10.00 D, no patients with hyperopes greater than +4.00 D, no patients with cylinders greater than 4.00 D, no enhancements done less than 6 months from a previous surgery, no patients with keratometry greater than 48 D and no patients with keratometry less than 39 D.

33. The method of claim 21, further comprising, between steps (c) and (d), the step of determining whether further treatment or corrective surgery could improve post-treatment visual capability and performing further treatment or corrective surgery to optimize post-treatment visual capability before providing an award.

34. A method providing award for unexpected outcomes resulting from product use, comprising the steps of:
a. providing an option to purchase a warranty prior to or during product purchase, the warranty providing award for unexpected outcome;
b. offering various, pre-determined levels of award with the warranty, each level of award associated with a degree of loss, the degree of loss based on a degree of variation of actual outcome from expected outcome, wherein a warranty purchase creates a warranty event;
c. determining the degree of loss associated with the warranty event; and
d. providing the level of award associated with the determined degree of loss.

35. A computer-readable medium that configures a computer system to perform a method for compensating loss resulting from unexpected outcome of service or unanticipated product performance, the method comprising the steps of:
a. providing an option to purchase a warranty prior to or during product purchase or service engagement, the warranty compensating loss resulting from the respective service provided or product purchased;
b. offering various, pre-determined levels of award with the warranty, each level of award associated with a degree of loss, wherein a warranty purchase creates a warranty event;
c. determining the degree of loss associated with the warranty event; and
d. providing the level of award associated with the determined degree of loss.

36. A computer-readable medium that configures a computer system to perform a method for providing award for unexpected medical outcomes, the method comprising the steps of:
a. providing an option to purchase a warranty prior to or during engagement of medical treatment, the warranty providing award for unexpected outcome;
b. offering various, pre-determined levels of award with the warranty, each level of award associated with a degree of loss, the degree of loss based on a degree of variation of actual outcome from expected outcome, wherein a warranty purchase creates a warranty event;
c. determining the degree of loss associated with the warranty event; and
d. providing the level of award associated with the determined degree of loss.

37. A computer-readable medium that configures a computer system to perform a method for compensating patients of lasik eye surgery that attain less than expected post-treatment visual capability, the method comprising the steps of:
a. providing an option to purchase a warranty prior to undergoing lasik eye surgery, the warranty providing award for less than expected post-treatment visual capability;
b. offering various, pre-determined levels of award with the warranty, each level of award associated with post-treatment visual capability, wherein warranty purchase initiates warranty coverage and award applicability;
c. determining post-treatment visual capability; and
d. providing the level of award associated with the post-treatment visual capability.

38. A computer-readable medium that configures a computer system to perform a method for providing award for unexpected outcomes resulting from product use, the method comprising the steps of:
a. providing an option to purchase a warranty prior to or during product purchase, the warranty providing award for unexpected outcome;
b. offering various, pre-determined levels of award with the warranty, each level of award associated with a degree of loss, the degree of loss based on a degree of variation of actual outcome from expected outcome, wherein a warranty purchase creates a warranty event;
c. determining the degree of loss associated with the warranty event; and
d. providing the level of award associated with the determined degree of loss.

39. A computer-readable medium that stores a program for compensating loss resulting from unexpected outcome of service or unanticipated product performance, the program comprising:
a. means for providing an option to purchase a warranty prior to or during product purchase or service engagement, the warranty compensating loss resulting from the respective service provided or product purchased;
b. means for offering various, pre-determined levels of award with the warranty, each level of award associated with a degree of loss, wherein a warranty purchase creates a warranty event;
c. means for determining the degree of loss associated with the warranty event; and
d. means for providing the level of award associated with the determined degree of loss.

40. A computer-readable medium that stores a program for providing award for unexpected medical outcomes, comprising:
a. means for providing an option to purchase a warranty prior to or during engagement of medical treatment, the warranty providing award for unexpected outcome;
b. means for offering various, pre-determined levels of award with the warranty, each level of award associated with a degree of loss, the degree of loss based on a degree of variation of actual outcome from expected outcome, wherein a warranty purchase creates a warranty event;
c. means for determining the degree of loss associated with the warranty event; and
d. means for providing the level of award associated with the determined degree of loss.

41. A computer-readable medium that stores a program for implementing a warranty program to compensate patients of lasik eye surgery that attain less than expected post-treatment visual capability, comprising:
a. means for providing an option to purchase a warranty prior to undergoing lasik eye surgery, the warranty providing award for less than expected post-treatment visual capability;
b. means for offering various, pre-determined levels of award with the warranty, each level of award associated with post-treatment visual capability, whereby a warranty purchase initiates warranty coverage and award applicability;
c. means for determining post-treatment visual capability; and
d. means for providing the level of award associated with the post-treatment visual capability.

42. A computer-readable medium that stores a program for providing award for unexpected outcomes resulting from product use, comprising:
a. means for providing an option to purchase a warranty prior to or during product purchase, the warranty providing award for unexpected outcome;
b. means for offering various, pre-determined levels of award with the warranty, each level of award associated with a degree of loss, the degree of loss based on a degree of variation of actual outcome from expected outcome, wherein a warranty purchase creates a warranty event;
c. means for determining the degree of loss associated with the warranty event; and
d. means for providing the level of award associated with the determined degree of loss.
